(19) United States
(12) Patent Application Publication
(10) Pub. No.: US 2005/0171613 A1
(43) Pub. Date: Aug. 4, 2005

(54) Wrist articulation prosthesis and set of elements allowing building of this prosthesis

(76) Inventors: Christian Sartorius, Meylan (FR); Adil Trabelsi, Villeneuve Lez Avignon (FR); Jean-Jacques Martin, Bourg En Bresse (FR)

Correspondence Address: YOUNG & THOMPSON, 745 SOUTH 23RD STREET, 2ND FLOOR, ARLINGTON, VA, 22202, US

(21) Appl. No.: 10/766,898

(22) Filed: Jan. 30, 2004

(51) Int. Cl. A61F 2/42

(52) U.S. Cl. 623/21.13; 623/23.44

(57) ABSTRACT

The prosthesis includes a main element wherein one part forms an articulation side, an element called the “radial” element and an intermediate sliding element. The articulation side that delimits the part of the main element is convex and presents a round form. The intermediate element forms a concave articulation side appropriate for cooperating with the convex articulation side that forms the main element, this concave articulation side presenting, in sectional view in the sagittal plane, that is in the plane in which the flexion and extension movement of the wrist takes place, a rounded circle form very slightly larger than that of the convex articulation side, and presenting, in sectional view in the frontal plane, that is in the plane in which the abduction and adduction of the hand takes place, a rounded circle form markedly larger than that of the convex articulation side.

### FIELD OF THE INVENTION

The present invention relates to a wrist articulation prosthesis and a set of elements allowing this prosthesis to be built.

### DESCRIPTION OF THE RELATED PRIOR ART

Such a prosthesis generally comprises three elements, that is to say two elements designed to be implanted in the extremity of the radius and in the large bone respectively, and one intermediate sliding element, interposed between the two bone elements for allowing the bone elements to slide one with relation to the other.

Existing articulation prostheses have the disadvantage of not completely restoring anatomical movement of this complex articulation, which is the wrist articulation.

Furthermore, positioning the bone element on the large bone involves an adequate resection of the latter as well as of the hemate bone, which has the disadvantages of increasing the duration of surgery and of weakening these two bones, which are most often in good condition.

Existing prostheses furthermore present the disadvantages of not always being sufficiently easy to manufacture or implant, and of involving considerable inventory management for hospital establishments.

The present invention aims to remedy this group of disadvantages.

### OBJECTS OF THE INVENTION

Its main objective is thus to provide a wrist articulation prosthesis that fully restores anatomical movement.

Another objective of the invention is to provide a prosthesis involving little or no resection of the large bone and the hemate bone for positioning the prosthesis.

Still another objective of the invention is to provide a prosthesis in which the design of the constituent pieces is optimized from the point of view of manufacturing and/or implantation of this prosthesis.

An additional objective of the invention is to provide a prosthesis that reduces the management of inventory of pieces that allow this prosthesis to be built.

### SUMMARY OF THE INVENTION

The prosthesis concerned comprises three elements such as the aforesaid, that is to say an element called the “main element” designed to be positioned on the large bone, an element called the “radial element” designed to be positioned on the radius, and an intermediate sliding element designed to be mounted on the radial element and to be interposed between this radial element and the main element for allowing clearance of these elements; the main element comprises a part forming an articulation side designed to abut against the large bone, and at least one bone support rod.

According to the invention,
The articulation side that delimits the said main element part is convex and presents a round form, particularly hemispherical or in a portion of a hemisphere, and
The intermediate element forms a concave articulation side appropriate for cooperating with the convex articulation side that forms the main element, this concave articulation side presenting, in sectional view in the sagittal plane, that is in the plane in which the flexion-extension movement of the wrist takes place, a rounded circle form very slightly larger than that of the said convex articulation side and presenting in sectional view in the frontal plane, that is in the plane in which the abduction-adduction of the hand takes place, a rounded circle form markedly larger than that of the said convex articulation side.

The convex articulation side that forms the main element allows the center of articulation to be positioned at the large bone, roughly at the same place as the anatomical center of articulation; this convex articulation side and the concave articulation side allowing a movement guided by pivoting in the case of the flexion-extension movement and a movement allowing a curvilinear displacement of the piece forming said convex articulation side with relation to the piece forming the said concave articulation side, in the case of the abduction-adduction movement.

This positioning of the prosthetic center of articulation and these possibilities for movement allow the anatomical articulation movement to be reliably restored.

Preferably, said bone support rod of the main element is laterally offset with relation to the said part forming an articulation side, so as to extend, when the main element is positioned on the large bone, along the lateral side of this large bone, that is the side of the latter that is not turned to the radius or to the metacarpal bones, this bone support rod presenting a length such that the rod may be inserted in a metacarpal bone in order to ensure that the main element is held in position with relation to the large bone.

Because of this bone support rod, the main element only necessitates a light resurfacing of the side of the large bone designed to receive the said part forming an articulation side, and does not involve significant resection of the large bone or of the hemate bone for its positioning.

Each bone support rode preferably forms an angle on the order of fifteen degrees with the perpendicular to a plane containing the side of the said part forming an articulation side designed to come into contact with the large bone.

The main element may only comprise a single bone support rod. Preferably, however, this element comprises two bone support rods, wherein one is designed to be inserted in the second metacarpal bone and wherein the other is designed to be inserted in the third metacarpal bone.

The rod designed to be inserted in the second metacarpal bone, may, in this case, present a bent form that adequately offsets the rod with relation to the said part forming an articulation side, while the pin designed to be inserted in the third metacarpal bone is rectilinear.

Advantageously, said part forming an articulation side is hollow at its area designed to come into contact with the large bone, and delimits a cavity allowing the part to be engaged on this large bone.

The abutting of this part at the large bone completes the anchoring that allows the said bone support rod.

The set of elements according to the invention may comprise a plurality of main elements of different sizes, adapted to the different sizes that may be presented depending on the articulation bones of the patients to be treated.

The set of elements according to the invention advantageously comprises a plurality of intermediate elements of different thicknesses, allowing the positioning of the large bone with relation to the “radial” element to be adjusted in order to obtain an adequate ligament tension.

This “radial” element advantageously comprises an extremity in the form of a platform abutting against the resected extremity of the radius, this extremity in the form of a platform delimiting a shoulder abutting against the extremity of the radius and a mounting side of the intermediate element.

According to a preferred embodiment of the invention, the mounting means that comprise the radial element and the intermediate element for mounting the intermediate element on the radial element comprise a dovetailed rib and a dovetailed groove appropriate for being closely engaged on the said rib, the “radial” element or the intermediate element comprising at least one rib or one projecting boss while, respectively, the intermediate element or the radial element comprises at least one corresponding rib or one cavity appropriate for receiving this rib or this boss with a locking mechanism.

Assembling the intermediate element and the radial element is thus performed in a particularly simple and rapid manner.

The “radial” element and the intermediate element, as well as the mounting means that comprise this radial element and this intermediate element for mounting the intermediate element on the radial element advantageously have a symmetrical form with relation to their median frontal plane.

A same “radial” element and a same intermediate element may be utilized for treating a right wrist or a left wrist, which very substantially simplifies inventory management of the pieces, allowing the prosthesis according to the invention to be built.

To be clearly understood, the invention is described again below with reference to the attached schematic drawing representing, by way of a non-limiting example, a preferred embodiment of the articulation prosthesis relating to the invention.

### BRIEF DESCRIPTION OF THE DRAWINGS

FIG. 1 is a perspective view while it is positioned on a wrist articulation, the bones of the hand and the extremity of the radius and of the ulna also being represented, the second and the third metacarpal bones as well as the extremity of the radius being shown in partial section;

FIG. 2 is a view of the prosthesis in perspective, before assembly;

FIG. 3 is a side view after assembly, an intermediate element that the view comprises being represented in partial section;

FIG. 4 is a side view of a main element that the view comprises;

FIG. 5 is a side view of this prosthesis, according to a direction perpendicular to the view according to FIG. 3, an intermediate element and the radial element that the view comprises being represented in partial section; and

FIG. 6 is a view of a detail visible on FIG. 5, at an enlarged scale.

### DETAILED DESCRIPTION OF THE PREFERRED EMBODIMENTS

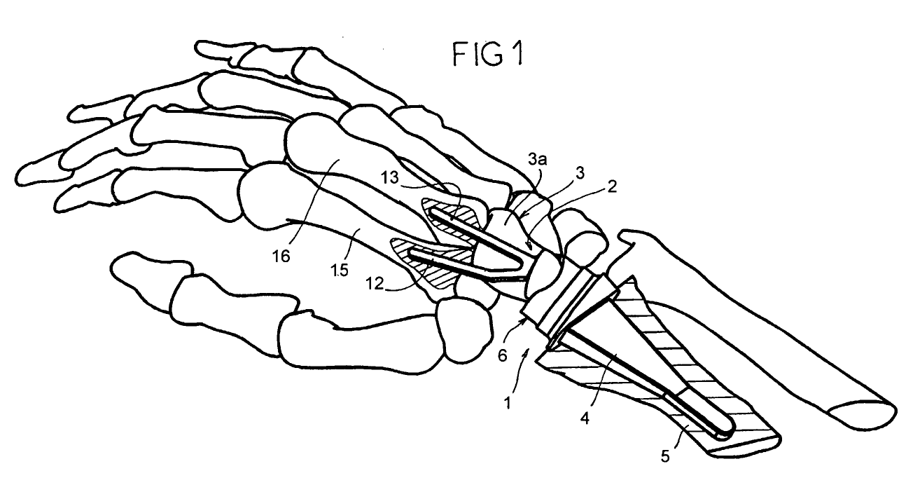

FIG. 1 represents a wrist articulation prosthesis 1.

This prosthesis 1 comprises three elements 2, 4, 6, that is to say an element 2, called “main,” designed to be positioned on the large bone 3, an element 4, called “radial,” designed to be positioned on the radius 5, and an intermediate sliding element 6 designed to be mounted on the radial element 4 and to be interposed between this radial element 4 and the main element 2 for allowing clearance of these elements 2 and 4.

As that appearing more particularly with reference to FIGS. 1 to 5, the main element 2 comprises a part 10 in a hemispherical form and two rods 12, 13 connected to the equatorial edge of this part 10.

The part 10 is hollow inside and delimits a cavity that opens in its equatorial edge. As is understood with reference to FIG. 1, this cavity is sized to allow the engagement of this part 10 on the large bone 3, after a light resurfacing of the corresponding portion of this large bone 3.

The outer side of the part 10 forms an articulation side appropriate for cooperating with an articulation side 30 described below, that forms the element 6.

The two rods 11, 12 form an angle of fifteen degrees with the perpendicular to the plane containing the equatorial edge of the part 10. The rod 12 presents a bend approximately one third of its length, counting from the part 10, that laterally offsets the rod with relation to this part 10. Rod 13 is rectilinear.

As well as that appearing in FIG. 1, the rods 12 and 13 present lengths such that the rods may be respectively inserted in the second metacarpal bone 15 and the third metacarpal bone 16 when the equatorial edge of the part 10 abuts against the large bone 3.

This insertion, combined with the engagement of a portion of the large bone 3 within the cavity that delimits the part 10, ensures that the main element 2 is held in position with relation to the large bone 3.

The aforesaid shaping of the rods 12 and 13 and their lateral offset with relation to the part 10 allows them in effect to extend, when the main element 2 is positioned on the large bone 3, along the lateral side 3a of this large bone 3, not turned toward the radius 5 or toward the metacarpal bones 15,16.

The radial element 4 comprises a rod 20 designed to be engaged in the radius 5 after suitable resection of the latter, and a proximal extremity 21 enlarged in the frontal plane, that is, the plane in which the abduction-adduction of the hand takes place, forming a platform.

This extremity 21 delimits a shoulder 22 for abutting against the extremity of the radius 5 and a proximal side 23 for receiving the intermediate element 6. The extremity presents, seen in the said frontal plane, a “wedge shaped” profile in such a way as to suitably orientate the shoulder 22 with relation to the side 23, and thus the element 6 with relation to the radius 5.

The element 4 also comprises a rib 25 in the form of a dovetail making a projection on the side. This rib 25 comprises a rib 26 projecting from its upper side of the extremity, orientated with relation to the said frontal plane.

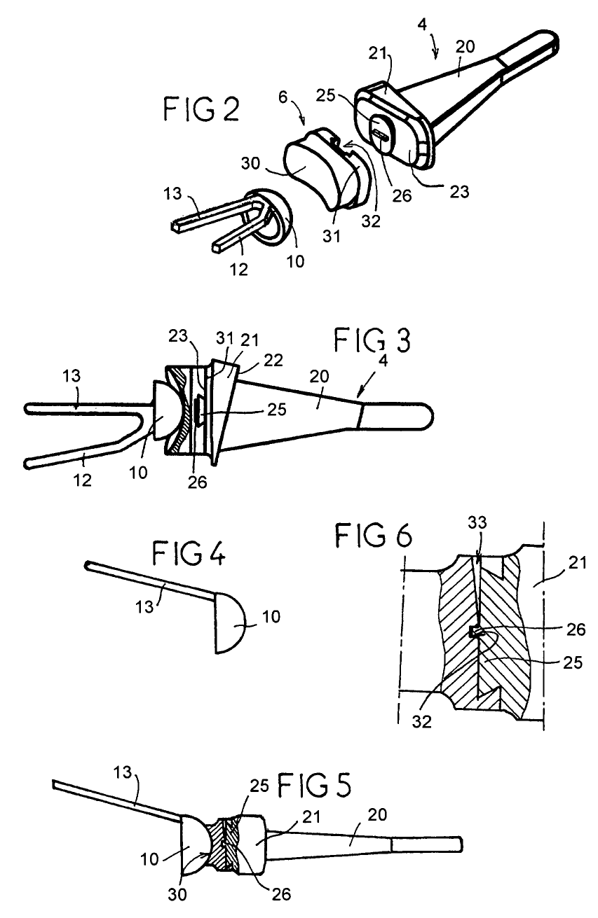

As well as that appearing in FIGS. 2, 3 and 5, the assembly of the element 4 presents a symmetry in form with relation to its median frontal plane, so that a same element 4 may be utilized on a right radius or a left radius.

The element 6 is in a material that promotes sliding, in particular a high-density polyethylene, while the elements 2 and 4 are metallic.

As shown in the figures, the element 6 forms a concave articulation side 30 designed to cooperate with the convex articulation side that forms the part 10 of the element 2.

This side 30 presents, in sectional view of the sagittal plane, that is in the plane in which the flexion and extension movement of the wrist takes place, a rounded circle form very slightly larger than that of the side that forms the part 10 (cf. FIG. 5), and presents, in sectional view in the frontal plane, a rounded circle form markedly larger than that of this side that forms part 10 (cf. FIG. 3).

Element 6 also presents, from the side opposite from the side 30, a flat side 31 designed to be received against the side 23 of the element 4.

Element 6 comprises a dovetailed groove 32 opening in this side 31 and a groove 33 oriented with relation to the frontal plane, opening in the bottom of this groove 32.

The groove 32 is sized to allow the tight engagement of the element 6 on the rib 25, and the groove 33 is shaped to receive the rib 26 with a locking mechanism in order to the immobilize the element 6 with relation to the element 4. The groove 33 comprises, as well as that appearing in FIG. 6, a bottom inclined from the side of its entrance, that forms a ramp promoting this locking mechanism.

The assembly of the element 6 presents a symmetry in form with relation to its median frontal plane, so that, as for element 4, a same element 6 may be utilized for a right or left articulation.

The elements 2 and 6, as well as possibly element 4, make up a set of elements allowing the building of the prosthesis 1, which comprises a plurality of main elements 2 or radial elements 4 of different sizes, adapted to the different sizes that may be presented depending on the articulation bones of the patients to be treated, and a plurality of intermediate elements 6 with different thicknesses, allowing the positioning of the large bone 3 with relation to a “radial” element 4 to be adjusted in order to obtain adequate ligament tension.

As well as that appearing previously, the invention contributes a material improvement to the prior art, by providing a wrist articulation prosthesis that completely restores anatomical movement. This prosthesis also involves little or no resection at the large bone and the hemate bone for its positioning, has constitutive pieces whose design is optimized from the point of view of manufacturing and/or implantation and allows inventory management of the pieces constituting this prosthesis to be reduced at hospital establishments.

The aforesaid set of elements allows, with reference to itself, pieces with dimensions fully adapted to the articulation to be treated to be provided.

It goes without saying that the invention is not limited to the embodiment described above by way of example but that it embraces on the contrary all variations of embodiments entering into the scope of protection defined by the attached claims.

1. A wrist articulation prosthesis, comprising three elements that is to say an element called the “main element”, designed to be positioned on the large bone, and element called the “radial element”, designed to be positioned on the radius, and an intermediate sliding element, designed to be mounted on the radial element and for being interposed between this radial element and the main element to allow clearance of these elements; the main element comprises a part forming an articulation side, designed to abut against the large bone, and at least one bone support rod
The prosthesis characterized in that:
The articulation side that delimits the said part of the main element is convex and presents a rounded form, particularly hemispherical or in a portion of a hemisphere, and
The intermediate element forms a concave articulation side appropriate for cooperating with the convex articulation side that forms the main element, this concave articulation side presenting, in a sectional view in the sagittal plane, that is in the plane in which the flexion and extension movement of the wrist takes place, a rounded circle form very slightly larger than that of the said convex articulation side, and presenting, in sectional view in the frontal plane, that is in the plane in which the abduction and adduction of the hand takes place, a rounded circle form markedly larger than that of the said convex articulation side.

2. The prosthesis according to claim 1, characterized in that the said bone support rod of the main element is laterally offset with relation to the said part forming an articulation side, so that the rod extends, when the main element is positioned on the large bone, along the lateral side of this large bone that is the side of the latter not turned toward the radius or toward the metacarpal bones, this bone support rod presenting a length such that the rod may be inserted in a metacarpal bone in order to ensure the main element is held in place with relation to the large bone.

3. The prosthesis according to claim 2, characterized in that each bone support rod forms an angle on the order of fifteen degrees with the perpendicular to a plane containing the side of the said part forming an articulation side designed to come into contact with the large bone.

4. The prosthesis according to claim 2, characterized in that the main element comprises two bone support rods, wherein one is designed to be inserted in the second metacarpal bone and wherein the other is designed to be inserted in the third metacarpal bone.

5. The prosthesis according to claim 4, characterized in that the rod designed to be inserted in the second metacarpal bone presents a bent form which offsets the rod with relation to the said part forming an articulation side, while the rod designed to be inserted in the third metacarpal bone is rectilinear.

6. The prosthesis according to claim 1, characterized in that the said part of the main element forming an articulation side is hollow at its area designed to come into contact with the large bone, and in that the part delimits a cavity allowing the engagement of this part on the large bone.

7. A prosthesis according to claim 1, characterized in that the radial element comprises an extremity in the form of a platform abutting against the resected extremity of the radius, this extremity in the form of a platform delimiting a shoulder abutting against the extremity of the radius and a side for mounting the intermediate element.

8. The prosthesis according to claim 1, characterized in that the mounting means that comprise the radial element and the intermediate element for mounting the intermediate element on the radial element comprise a rib in the form of a dovetail and a groove in the form of a dovetail appropriate for being closely engaged on the said rib, the radial element or the intermediate element comprising at least one rib or one projecting boss while, respectively, the intermediate element or the radial element comprises at least one corresponding groove or one cavity appropriate for receiving this rib or this boss with a locking mechanism.

9. The prosthesis according to claim 1, characterized in that the radial element and the intermediate element, as well as the mounting means that comprise this radial element and this intermediate element for mounting the intermediate element on the radial element, have a symmetrical form with relation to their median frontal plane.

10. A set of elements allowing the prosthesis to be built according to claim 1, characterized in that the set comprises a plurality of main elements of different sizes, adapted to the different sizes that may be presented depending on the articulation bones of the patients to be treated.

11. The set of elements according to claim 10, characterized in that the set comprises a plurality of intermediate elements with different thicknesses, allowing the positioning of the large bone with relation to the radial element to be adjusted in order to obtain an adequate ligament tension.
